(19) United States
(12) Patent Application Publication
(10) Pub. No.: US 2004/0171933 A1
(43) Pub. Date: Sep. 2, 2004

(54) Mammography needle biopsy system and method

(76) Inventors: Milton Stoller, West Hartford, CT (US); Anthony Scandura, Scotland, CT (US); Kenneth F. De Freitas, Patterson, NY (US)

Correspondence Address: Paul Teng, Esq., Cooper & Dunham LLP, 1185 Avenue of the Americas, New York, NY, 10036, US

(21) Appl. No.: 10/723,406

(22) Filed: Nov. 25, 2003

Related U.S. Application Data
(60) Provisional application No. 60/428,884, filed on Nov. 25, 2002.

(51) Int. Cl. A61B 5/05; A61B 19/00

(52) U.S. Cl. 600/427; 606/130; 378/37; 128/920

(57) ABSTRACT

A breast needle biopsy system and method use two-dimensional information about an abnormality from a scout view of the breast of a prone or upright patient but only one-dimensional information from each of one or more stereo views to control a needle guidance stage relative to an abnormality in the breast. An additional feature is to provide a live display of a least distance between calculated positions or loci of positions of a target in the breast to enable a health professional to make new selections of coordinates from the scout and/or stereo views in an effort to reduce the least distance, or to otherwise account for that distance. The live display of a least distance also improves existing systems that rely on two-dimensional information from each of two stereo images to calculate an abnormality's position in the breast.

### FIELD

This patent specification is in the field of mammography and more specifically in the field of using x-ray imaging of a breast to guide procedures such as needle biopsy.

### BACKGROUND

Stereotactic needle biopsy systems have been used in this country and elsewhere for a number of years. Typically they take two stereo x-ray images of a compressed and immobilized breast, the health professional identifies the coordinates of an abnormality seen on each image, and the system uses these coordinates and knowledge of system geometry to calculate a path for the biopsy needle and guide a needle stage. The patient is prone in some systems and seated in others.

For example, the assignee of this patent application sells a system tradenamed MultiCare for use in localization of previously discovered abnormalities such as lesions and microcalcifications in the breast, and guidance in procedures such as large core biopsy, needle core biopsy, wire localization, vacuum assisted biopsy and fine needle aspiration. As illustrated in FIG. 1 (not to scale), the MultiCare system includes a patient table 100 on which the patient rests in a prone position, with a breast protruding downward. The breast is compressed and immobilized between a breast platform 102 and a movable compression plate 104, all fitting between an x-ray source 106 and an x-ray receptor 108 mounted on a C-arm 110 that pivots about a vertical axis, relative to platform 102 and plate 104, to image the breast compressed between them from different angles. X-ray receptor 108 is a filmless, digital receptor that directly generates a digital x-ray image, without relying on film. Typically, three different x-ray images are taken while the breast remains compressed and immobilized—a scout image that typically, but not necessarily is at 0° (with a central axis of the x-ray beam perpendicular to the plane of the compressed breast, i.e., to the breast platform and the compression plate), and two stereo images that typically, but not necessarily, are at +15°. The scout image, typically is used to verify that the abnormality is approximately centered within a biopsy window in the compression plate, and only the two stereo views are used to derive, from each, two-dimensional information about the abnormality as seen in the respective stereo view, so that the two sets of coordinate pairs can then be used to find the position of the abnormality in three-dimensional space. This three-dimensional information in turn is used to partly or fully automatically position a needle for insertion into the breast and guide the insertion. Prior art systems also had the ability to substitute the scout view for one of the stereo views and thus derive two-dimensional information from each of the scout view and stereo view, and use the resulting two sets of coordinate pairs to calculate three-dimensional information about the abnormality. Another pair of stereo images may be taken with a needle in place in the breast, to confirm its position relative to the abnormality of interest. FIG. 2 illustrates a version of the same system in a perspective view. In addition to the same or similar patient table 100 and C-arm 110 carrying x-ray source 106 and x-ray receptor 108, FIG. 2 illustrates a needle guidance stage assembly 200, a control console 202 and a workstation 204. Console 202 contains x-ray control electronics and a high voltage generator, and has pushbuttons to modify exposure control menus shown on its screen. An internal computer controls the x-ray operations, and a display screen show the status of these operations and other information. Workstation 204 is an image capture and enhancement system that includes an image receiving subsystem, a computer, a high resolution monitor 206, and interface devices such as a keyboard 208 and a trackball 210. Workstation 204 displays the stereo images taken typically at ±15°, and the health professional marks points thereon using a cursor controlled through the interface devices. The workstation then uses the information to calculate the three-dimensional position of the abnormality of interest and to send resulting information to needle guidance stage assembly 200 as well as to display information to the user.

Additionally, the assignee of this patent also sells a system tradenamed StereoLoc which performs similarly to the MultiCare system, where the patient remains upright, either seated or standing, and not in the prone position. As illustrated in FIG. 11 (not to scale), in the StereoLoc system, the breast is compressed and immobilized between a breast platform 102 and a movable compression plate 104, all fitting between an x-ray source 106 and an x-ray receptor 108 mounted on a C-arm 110 that pivots about a vertical axis, relative to platform 102 and plate 104, to image the breast compressed between them from different angles. X-ray receptor 108 is a filmless, digital receptor that directly generates a digital x-ray image, without relying on film.

Commonly assigned U.S. Pat. Nos. 5,289,520, 5,426,685, and 5,609,152, and Operator's Manual 9-500-0250, Revision 1, copyright 1999, from the assignee, discuss in more detail various aspects of the MultiCare system and stereotactic systems in general, and are hereby incorporated by reference in this patent specification. Another system is sold by Fischer Imaging Corporation under the name Mammotest. U.S. Pat. Nos. 5,078,142, 5,240,011, 5,415,169, 5,735,264, 5,803,912, and 6,022,325 are said to be related. Unlike the current MultiCare system that uses a digital x-ray receptor and provides an essentially instantaneous display of the x-ray images, some earlier systems took x-ray film images, thus requiring time to develop the film and place it on a light box for viewing and marking by the health professional while the patient's breast remains compressed and immobilized, with attendant discomfort. The file history of the first-filed patent among those said to pertain to the Mammotest system sold by Fischer Imaging Corporation (U.S. Pat. No. 5,078,142) contains an IDS and a Form PTO-1449 in which the applicants acknowledge as prior art an earlier system called TRC Mammotest System and identify four documents pertaining to that earlier system. The TRC Mammotest System also pertains to needle biopsy of a prone patient's breast.

EP 0 390 652 also discusses a stereotactic breast x-ray system and illustrates an example in which the stereo images of the breast are taken on film that appears to be in a horizontal orientation. Published European Patent Application 0 146 511 appears to be similar, and a similar unit called Cytoguide is illustrated in a Product Information brochure by SMITH RÖNTGEN distributed by Technomed USA of Bay Shore, New York. Stereotactic needle biopsy is discussed in Dowlatshani, K., The needle replaces the knife, Breast Care, June 1989.

### SUMMARY

The known systems require the health professional to identify the location of the abnormality on each stereo image in two dimensions, e.g., the x-y coordinates on each stereo image, to thereby provide the information needed to calculate the three-dimensional location of the abnormalities in space and thus guide needle insertion. While this conforms to and uses geometry principles known well before the development of these systems, and has worked well for a long time, it has been discovered that significantly better results can be achieved in a completely different way.

In particular, it has been discovered that superior results can be achieved by using a scout image to derive two-dimensional information regarding the abnormality, and using one or more stereo images to derive one-dimensional information regarding the same abnormality. For example, the new approach identifies the x-y coordinates of an abnormality in a scout image, but only the x-coordinate in one or more images taken at different angles.

The scout image, which to the inventors' knowledge was only used in the known systems to generally verify that the abnormality is approximately centered within the biopsy window, is used in the new approach in a very different way makes it possible to need only one-dimensional information from one of the stereo views in order to calculate the position of the abnormality or a target related thereto in three dimensions. The scout image can be the clearest image of all because for the scout image the x-rays pass through a lesser thickness of breast tissue as compared to the prior art's stereo images. Moreover, the scout image matches more closely the images health professionals use to diagnose abnormalities, and can be more reliable in visualizing and identifying the abnormality and its characteristics. Further, the scout image is in a plane that can be perpendicular to the path of the needle, and thus the needle penetration path can be more easily visualized. A view at another angle (a stereo view) is used in the new approach not for two-dimensional information but for one-dimensional information, e.g., to identify a plane that passes through the origin of the x-rays and through the abnormality. This plane intersects the stereo image at a line and its position in the stereo image can be defined by a single dimension. This single dimension does not specify a line through the x-ray origin and the abnormality, as such a line would intersect the stereo image at a point that needs two dimensions to specify its location. Such a line is specified by the two-dimensional information from the scout view, and intersects the scout view at a point.

Because only the scout view and one additional (stereo) view need to be taken and used in an example of the new approach, the health professional has the option to acquire only two images, thus speeding up the procedure as compared with traditional techniques of taking a scout view to see if the compression plate and its biopsy window are properly positioned relative to the abnormality and then taking two more (stereo) views for use in localizing the abnormality in three-dimensional space and using this localization to guide the needle. Taking and using only two images subjects the patient to a lower x-ray dose and speeds up the process because the health professional does not need to reposition the x-ray equipment for a second stereo view and does not have to analyze and mark a third view. A second stereo image (for a total of three views) can be taken and used if the abnormality cannot be seen well in the first stereo image, or for other purposes.

If two stereo images are used in the new approach, in addition to the scout view, still solely one-dimensional information needs to be taken from each stereo image. However, the information from the second stereo image can be used to refine the process of selecting a needle path, as explained below.

If two stereo images are used in the known manner so that a location in each is identified in two dimensions, the calculation of the three-dimensional location of the abnormality can be conceptualized as identifying the locations in space of two lines, each passing through the abnormality but one also passing though the x-ray origin's position in space when the first image was taken and the other through the origin's position when the other image was taken. Ideally, the two lines would intersect in three-dimensional space, but in practice they may not. One way to resolve the ambiguity resulting from non-intersection is to find the closest distance in space between the lines and postulate that the center of the abnormality or a target point is at the midpoint. This can provide benefits, but in the known system the health professional may not be aware that it is being done, and certainly may not be aware of the actual distance between the lines. It has been discovered that such information can be helpful if timely available and displayed to enable the health professional to take it into account, e.g. by selecting an image coordinate that reduces the spacing between the two lines, or in some other way. This applies to known systems, and similar considerations also apply to the new approach to finding the abnormality's three-dimensional location in space.

Where each of two or more stereo views is being used to generate two-dimensional coordinates, each of the stereo views is taken at a different angle, one of which may be, but need not be, perpendicular to the breast plate and the breast platform.

In a first example of the new approach only a scout image and a single stereo image are taken and used. Conceptually, the scout image provides information that together with information about system geometry identifies a line in space that passes through the abnormality and the origin of the x-rays in its position when the scout view was taken. The single stereo view is used solely to provide one-dimensional information that defines not the location of the abnormality but a conceptualized vertical plane (not a line) that intersects the abnormality and the x-ray origin in its position in space when the stereo view is taken. Again conceptually, the intersection of the line and the plane define the abnormality's three-dimensional position in space, or the position of a related target. (The term stereo view refers here to a view taken at an angle other than that for the scout view. The scout view, e.g. a 0° view, is at an image plane parallel, or nearly so, to the plane of the breast compressed between the breast platform and compression plate. Typically, but not necessarily, the x-rays have a central ray normal to the compressed breast, so a stereo view is a view taken at another angle, typically at 15° to a line normal to the compressed breast.) In a second example of the new approach two stereo views are used in addition to the scout view. The second stereo view defines a second vertical plane that passes through the abnormality and the position in space of the x-ray origin when the second stereo view was taken. The two conceptual planes intersect at a line that ideally would be parallel to the scout view image plane and ideally would intersect the line through the x-ray origin and the abnormality defined by the scout view. However, if the two lines do not intersect, the least distance between them can be taken as a locus of points related to the abnormality's three-dimensional location in space. One of these points, typically the midpoint along the shortest distance between the lines, can be taken as a center of the abnormality or as a target. Here it is particularly beneficial to use the additional feature of a live display of this least distance, or of related information, while the health professional is selecting a coordinate on an image. For example, the two-dimensional information from the scout view and the one-dimensional information from each of two stereo views can be used to calculate two lines in space, each passing through an estimated position of the abnormality in three-dimensional space. A live display of the distance between these two lines can allow the health professional to change the selection from one or more of the views to thereby in effect move one or both lines and thus change the calculated distance between these two lines. This can be an iterative process in which the health professional can go to different views to change previously selected coordinates and thus affect the calculation of the three-dimensional position in space of the abnormality or a target related thereto. Alternatively, the health professional can account for this distance in some other way.

Alternatively, in the second example the scout image and one of the stereo images can be used to define one target as in the first example, and the scout view and the second image can be used to define a second target using the same technique. If the two targets do not coincide, another point can be selected as the final target, e.g., a point on a line connecting them, such as the midpoint. Again, if information regarding the separation between the initially calculated targets is displayed to the health professional, it can serve as a basis for modifying the selection of coordinates to reduce the spacing or to otherwise make an informed decision on the biopsy procedure.

Additional targets can be defined on the basis of the targets calculated as discussed above, i.e. targets that are along the same needle path but at different depths in the breast, and targets that are spaced from the main target in the plane of the compressed breast.

Other features and benefits of the new approach should become apparent from the detailed description of preferred embodiments taken in conjunction with the drawing.

### BRIEF DESCRIPTION OF THE DRAWING

FIG. 1 illustrates a known system but also equipment for practicing preferred embodiments disclosed in this patent specification.

FIG. 2 is similar but illustrates the system in a perspective view and also illustrates additional components.

FIG. 3 illustrates geometry of a scout view and a stereo view.

FIG. 4 illustrates points of interest in the view of FIG. 3, in a common coordinate system.

FIG. 5 illustrates a display of a scout image and two stereo images, and of other information.

FIG. 6 illustrates a coordinate system used in selecting coordinates in displayed images.

FIG. 7 illustrates a coordinate system in an image plane.

FIG. 8 illustrates a needle stage coordinate system.

FIG. 9 illustrates zeroing of a needle stage.

FIG. 10 illustrates a manual input device (slider) for entering one-dimensional information regarding an abnormality or target.

FIG. 11 illustrates another known system modified to practice embodiments disclosed in this patent specification.

### DETAILED DESCRIPTION OF PREFERRED EMBODIMENTS

A first illustrative embodiment uses only a scout image and a single stereo image to calculate the location of an abnormality in three-dimensional space. Conceptually, the scout image and knowledge of system geometry help identify a line in space that passes through the abnormality and the origin of the x-rays at the time the scout view was taken. The single stereo view only helps identify a vertical plane that passes through the abnormality and the origin of the x-rays at the time the stereo view was taken. The intersection in space of the line and plane helps identify the location in three-dimensional space of the abnormality or of a target of interest. If two stereo images are available, the health professional can select for use in this embodiment the one that shows the abnormality more clearly or in a preferred orientation.

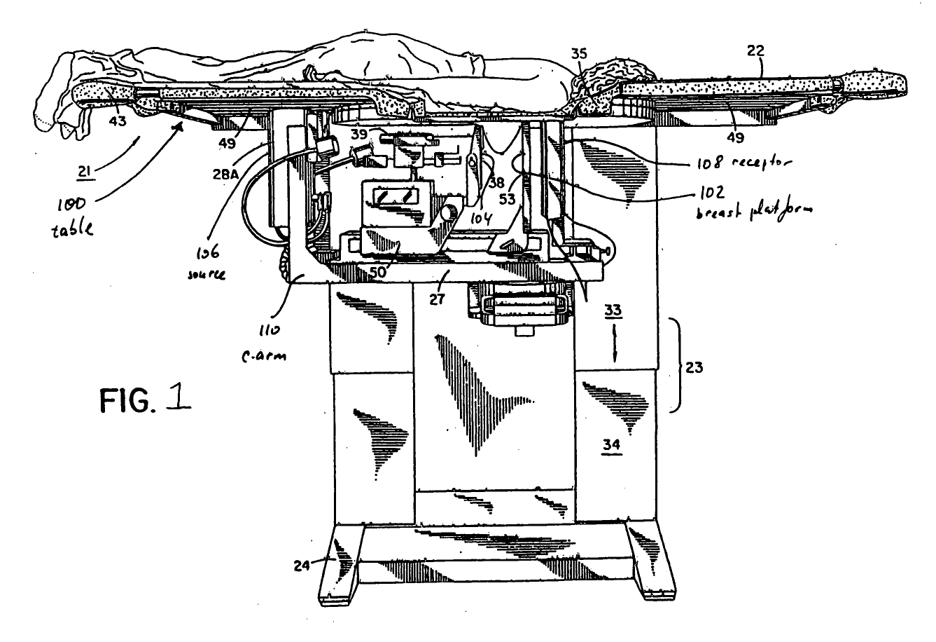

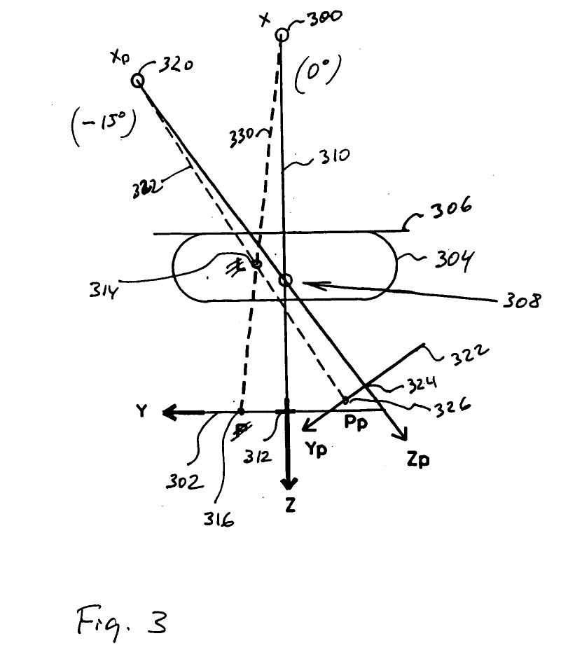

While the explanation above may be useful in conceptualizing the new processes, in actual practice the parameters derived and the calculations can be different. In order to better appreciate a practical implementation, it would help to define the various coordinate systems that might be used given a geometry of a particular hardware implementation. To this end, FIG. 3 illustrates in schematic form a geometry for taking two different x-ray views—a scout view and a −15° stereo view. FIG. 3 conforms to a horizontal section of the system illustrated in FIG. 1 but is only a schematic view. The section intersects the origin of the x-rays in source 106, the compressed breast, and the image plane in receptor 108, in their respective positions for each of the two views. For ease of illustration, the relative dimensions and angles in FIG. 3 are not to scale.

In FIG. 3, the x-ray origin for the scout view is at X) and the image plane is at 302, which may coincide with the plane of the phosphor in digital x-ray receptor 108, or with a functionally comparable plane if another type of a digital x-ray receptor is used, such as a flat plate receptor available from Direct Radiography Corporation of Newark, Del. A (respective) Cartesian coordinate system for each view can be used in this example, although other coordinate systems can be used instead, e.g., polar coordinates. For the scout view, the x-direction is out of the paper, and the y-direction and z-directions are indicated by respective arrows labeled Y and Z. The compressed and immobilized breast is schematically illustrated at 304. The surface of compression plate 104 that bears against breast 304 is at 306. The rotational center of C-arm 110 is at 308. A center ray 310 of the x-rays from origin 300 is perpendicular to image plane 302 and intersects it at 312. A line 330 intersects origin 300 and an abnormality 314 in breast 304, and intersects image plane 302 at 316 (P).

FIG. 3 also illustrates a stereo view that typically is taken at an angle of −15°, from an x-ray origin position at 320 (Xₚ), but in the drawing the angle is exaggerated for clarity of illustration. The image plane for this stereo view is at 322 (Yₚ). A line from origin 320 through the center of rotation at 308 intersects image plane 322 at 324, and the projection of abnormality 314 on image plane 322 is at 326 (Pₚ). The illustrated stereo view has its own coordinate system, in which the x-direction again is out of the paper, and the y-direction and the z-direction are along the respective arrows labeled Yp and Zp.

The points of interest can be defined in the respective coordinate systems of FIG. 3 of each view. However, it has been found to be convenient to transform them into a common coordinate system, as is done in the current MultiCare system for the points marked on two stereo views, using conventional trigonometric relationship and knowledge of hardware components. FIG. 4 illustrates the points of interest in such a common coordinate system. In the common coordinate system, abnormality or target or 314 has coordinates xyz, x-ray origin X or 300 for the scout view has coordinates x₁y₁z₁ and the projection P or 316 of the abnormality on the scout view image plane 302 has coordinates x₂y₂z₂, the x-ray origin for the stereo view Xp or 320 is at x₃y₃z₃, and the projection of abnormality 314 on the image plane of the stereo view is marked with a line by the operator, this line being defined by any two points thereon on the phosphore, one at x₄y₄z₄ and the other at x₅y₅z₅. Note that only the x-value for point 326 in the coordinate system for the stereo view need be derived from the stereo image, not two-dimensional information regarding that point. The y-information from the scout image, suitably transformed, can be used to supply a y-value related to that point in the common coordinate system of FIG. 4. The z-values in the common coordinate system (except for the target 314) can be computed from the xy values and the known distance between the x-ray origin and the image plane, using known trigonometric relationships.

Using the common coordinate system of FIG. 4, relevant geometry can be described by two sets of equations, one for line 330 from x-ray origin 300 to projection 316 of abnormality 314, and one for the plane 322 that is perpendicular to the sheet of FIGS. 3 and 4 and is defined by x-ray origin 320 and the projection 326 of the same abnormality 314:

The equation for the line is:

$$\frac{x - {x}_{1}}{{x}_{2} - {x}_{1}} = \frac{y - {y}_{1}}{{y}_{2} - {y}_{1}} = \frac{z - {z}_{1}}{{z}_{2} - {z}_{1}}$$

The equation for the plane is:

$$\vert \begin{matrix}{y}_{4} - {y}_{3} & {z}_{4} - {z}_{3} \\ {y}_{5} - {y}_{3} & {z}_{5} - {z}_{3}\end{matrix} \vert * ( x - {x}_{3} ) + \vert \begin{matrix}{z}_{4} - {z}_{3} & {x}_{4} - {x}_{3} \\ {z}_{5} - {z}_{3} & {x}_{5} - {x}_{3}\end{matrix} \vert * ( y - {y}_{3} ) + \vert \begin{matrix}{x}_{4} - {x}_{3} & {y}_{4} - {y}_{3} \\ {x}_{5} - {x}_{3} & {y}_{5} - {y}_{3}\end{matrix} \vert * ( z - {z}_{3} ) = 0$$

The unknown three-dimensional position of the abnormality or target 314 is defined by coordinates xyz in the common coordinate system.

One way to find the unknowns is through a computer program such as illustrated below, written in Visual Basic, where:

Points (x1, y1, z1) and (x2, y2, z2) are the endpoints of line 330 in FIG. 4 (i.e, points x₁y₁z₁ and x₂y₂z₂); and

Points (x3, y3, z3), (x4, y4, z4), and (x5, y5, z5) are points that define the vertical plane 322 normal to the paper of FIG. 4 that coincides with points x₃y₃z₃, x₄y₄z₄, and x₅y₅z₅).

Point (x, y, z) is the point whose coordinates as sought.

Visual Basic Program:

```
Option Explicit
Dim x1, x2, x3, x4, x5, y1, y2, y3, y4, y5, z1, z2, z3, z4, z5 As Single
Dim a, b, c, d, e, f, g, h, j, x, y, z As Single
Private Sub cmdCalculate_Click( )
  x1 = Val(txtX4.Text)  ‘Endpoint 1 of line.
  y1 = Val(txtY4.Text)
  z1 = Val(txtZ4.Text)
  x2 = Val(txtX5.Text)  ‘Endpoint 2 of line.
  y2 = Val(txtY5.Text)
  z2 = Val(txtZ5.Text)
  x3 = Val(txtX1.Text)  ‘Point 1 of plane.
  y3 = Val(txtY1.Text)
  z3 = Val(txtZ1.Text)
  x4 = Val(txtX2.Text)  ‘Point 2 of plane.
  y4 = Val(txtY2.Text)
  z4 = Val(txtZ2.Text)
  x5 = Val(txtX3.Text)  ‘Point 3 of plane.
  y5 = Val(txtY3.Text)
  z5 = Val(txtZ3.Text)
  If ((x2 − x1) <= 0.01 Or ((x1 − x2) <= 0.01)) Then
    x2 = x1 + 0.02
  End If
  a = y4 * z5 − y3 * z5 − y4 * z3 − y5 * z4 + y3 * z4 + y5 * z3
  b = z4 * x5 − z3 * x5 − z4 * x3 − z5 * x4 + z3 * x4 + z5 * x3
  c = x4 * y5 − x3 * y5 − x4 * y3 − x5 * y4 + x3 * y4 + x5 * y3
  e = z2 − z1
  f = −x2 + x1
  g = x2 * z1 − x1 * z2
  h = y2 − y1
  j = x2 * y1 − x1 * y2
  x = (a * f * f * x3 + b * f * j + b * f * f * y3 + c * g * f +
    c * f * f * z3)_/(a * f * f − b * f * h − c * e * f)
  y = (−h * x − j)/f
  z = (−e * x − g)/f
  picResults.Cls
  picResults.Print “x=”; Round(x, 2)
  picResults.Print “y=”; Round(y, 2)
  picResults.Print “z=”; Round(z, 2)
End Sub
Private Sub cmdExit_Click( )
  End
End Sub
```

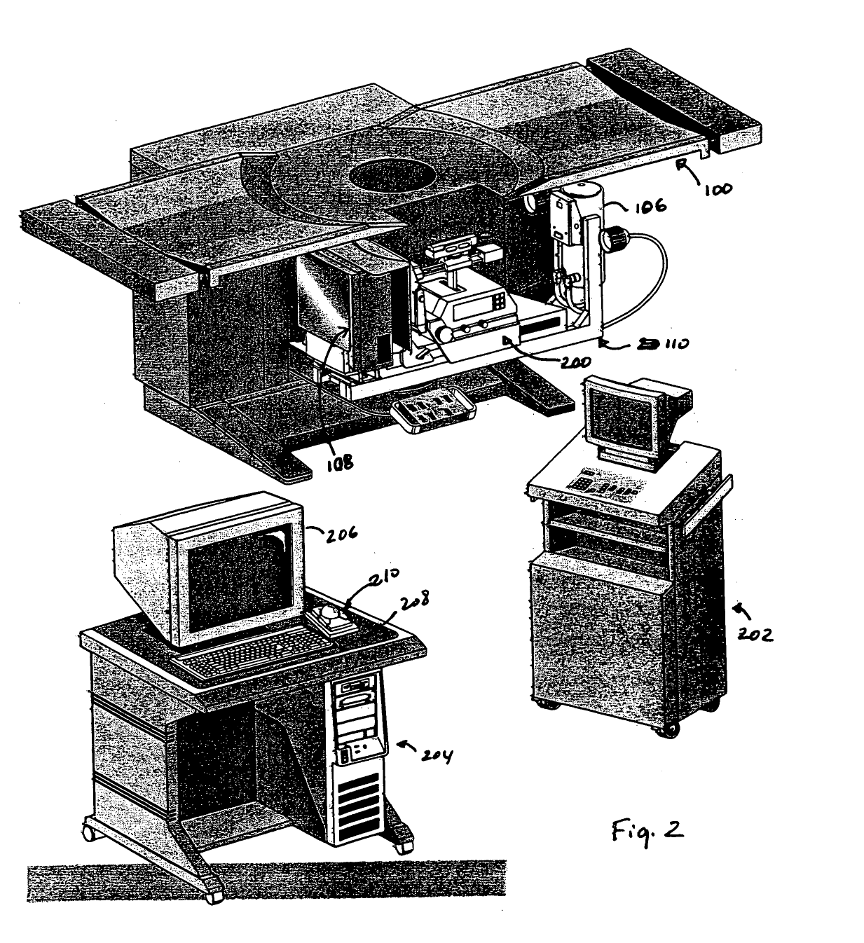

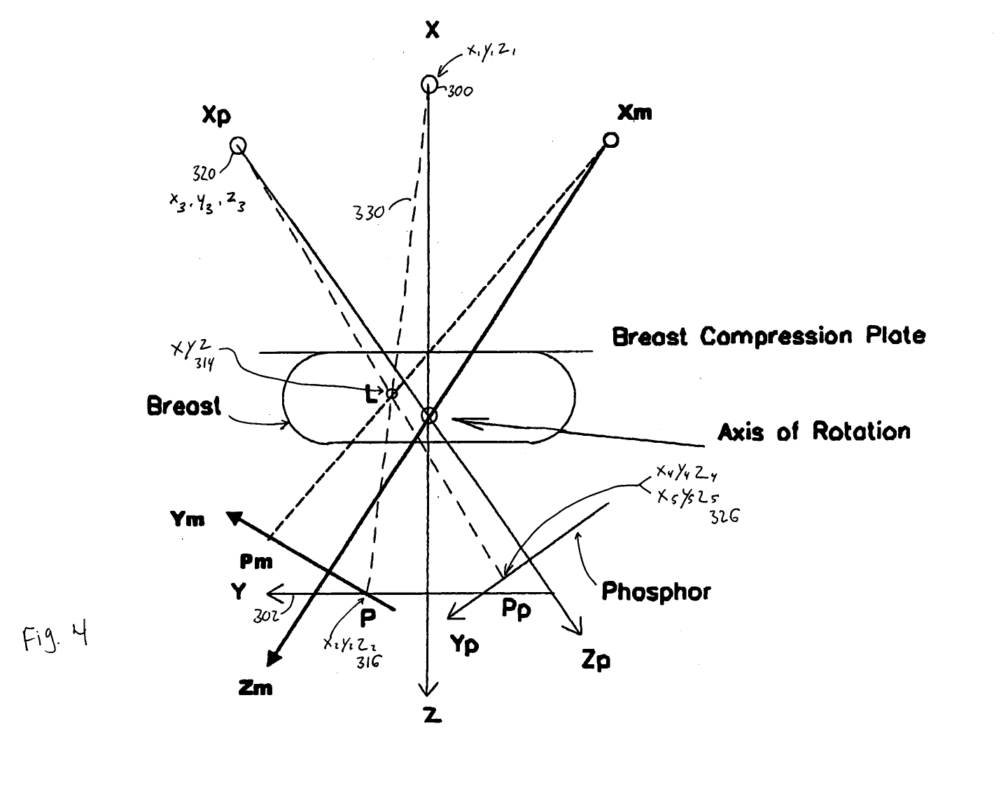

The information needed to define the conceptual line and plane comes from manual input based on a scout view and a stereo view. FIG. 5 illustrates schematically a display of three views on monitor 206 (FIG. 1), a scout view at 500 and two stereo views at 502 and 504 that may be taken at ±15. The views contain respective images 500a, 502a and 504a of the same abnormality, illustrated as points for simplicity although in fact the abnormality images would occupy areas in the respective views. Although two stereo views are illustrated, this embodiment only needs one, provided it shows the abnormality sufficiently clearly. To derive two-dimensional information about the abnormality from the scout image 500, the health professional uses a manual input device, such as trackball 210 (FIG. 2), a mouse, a joystick or some other input device, to position a targeting symbol 500b over the abnormality 500a. By clicking or otherwise indicating that targeting symbol 500b is on abnormality 500a, the health professional enters the two-dimensional information (xy coordinates of symbol 500b at the current position on image 500) into the computer in workstation 204. This part of the process is essentially identical to the entry of two-dimensional information from a stereo view in the current MultiCare system, but the sqftware is modified to account for the fact that the two-dimensional information comes from a scout view and will be used in a fundamentally different way.

FIG. 5 also illustrates two windows, 506 and 508, for displaying information to assist the health professional. Window 506 can be similar to a targeting dialog box displayed in the current MultiCare system, except that the information regarding coordinates in the system disclosed in this patent application comes from targeting based on the new way of finding target locations. Window 508 pertains to a display of the least distance between targets or conceptual lines calculated in different ways, and is discussed in more detail below.

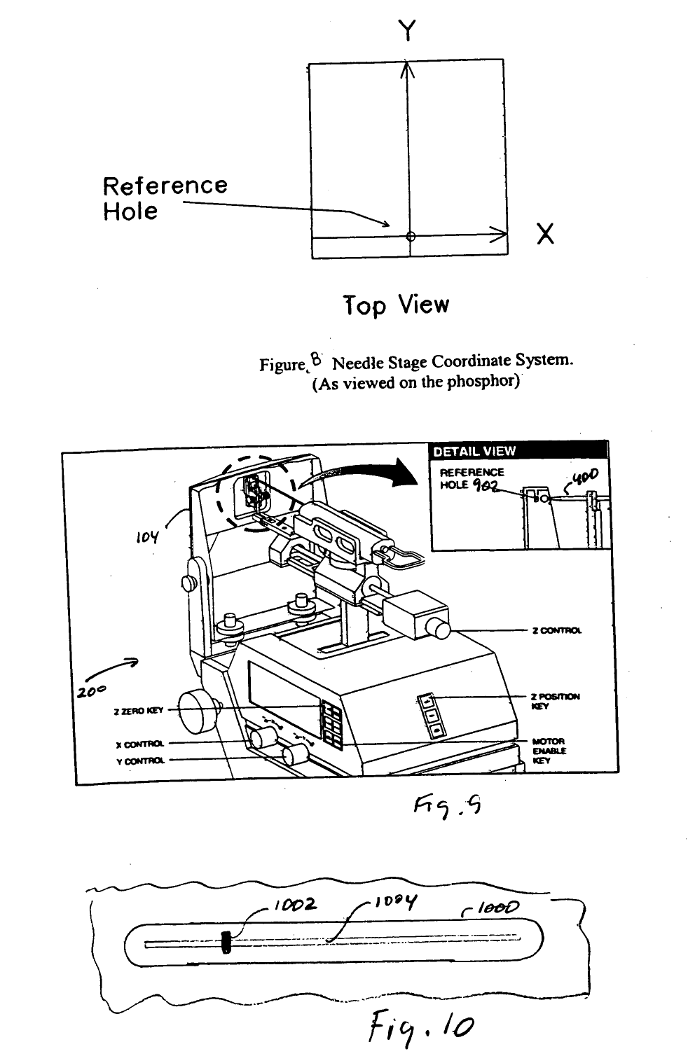

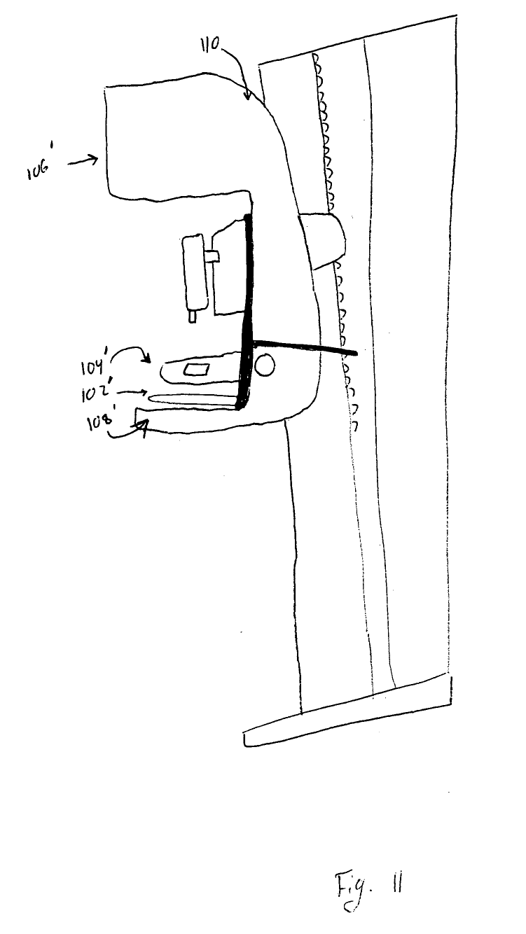

Using the same or a different input device, the health professional then moves a vertical line or stripe (not a cursor or some other symbol that identifies a point) displayed over a stereo image, for example vertical line 500b displayed over stereo image 502, until it is aligned with the abnormality image 502a, and clicks or otherwise indicates proper alignment. This enters one-dimensional information into the computer in workstation 204, related where vertical line 502b is in stereo image 502. This information from views 500 and 502 is sufficient for the first example of an embodiment discussed above. (A currently preferred input device is a slider separate from trackball 210 (FIG. 2), as discussed below regarding FIG. 10.)

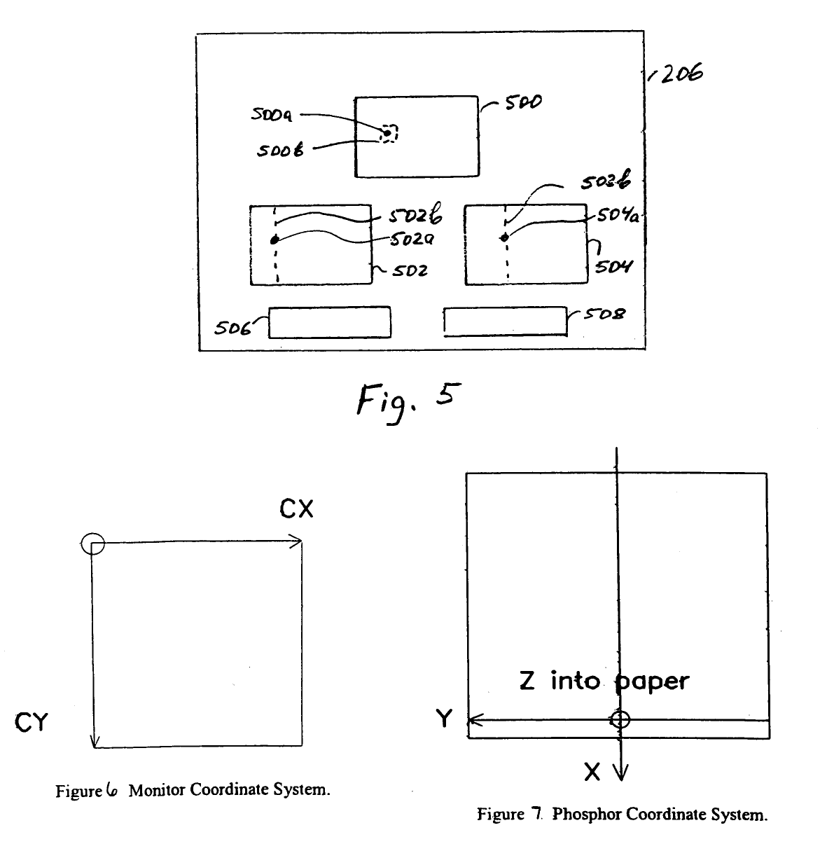

In practice, different coordinate systems may be used for different purposes, and thus coordinate conversions are carried out. The images displayed on monitor 206 (FIGS. 1 and 5) can conform to the xy coordinate system illustrated in FIG. 6, which the health professional uses to select coordinates in a displayed image. As in the current MultiCare system, the coordinates in image 500 can be in cursor units. They are converted to units of distance (millimeters), and are transformed into a coordinate system that relates to the respective image planes of receptor 108 in its positions for the different views (the image plane is at the phosphor in the currently used digital receptor, or at a comparable plane in different types digital receptors). The image plane (phosphor) coordinate system is illustrated in FIG. 7. In turn, the coordinates that are in the coordinate systems of the respective image planes are transformed into the common coordinate system of FIG. 4 used to calculate the abnormality's or target's position in space. Similarly, the x-coordinate (and only the x-coordinate) from stereo image 502 is transformed into the coordinate system of the image plane, then into the common coordinate system of FIG. 4 for the calculation.

In practice, the system also calculates the position in space of a reference hole in breast compression plate 104 (FIG. 1), as in the current MultiCare system, using calculations similar to those for target 314, so that the reference hole can serve as the zero calibration point for needle guidance stage 200. As a result, the system has information regarding the location of the target or abnormality relative to the reference hole. During calibration, the biopsy needle is physically moved to the center of the reference hole and the needle stage xyz values are zeroed out. Needle stage 200 has its own coordinate system, illustrated in FIG. 8, so the xyz coordinates of the abnormality found in the common coordinate system of FIG. 4 are transformed into the needle stage coordinate system of FIG. 8 for the actual positioning of the needle that determines where it will enter the breast and how far its should penetrate. The coordinate transformation processes and the use of the information regarding the three-dimensional positions in space of the abnormality and the reference hole to guide the needle stage are the same or similar to those in the current MultiCare system and thus need not be described in greater detail

The zero calibration of the needle is illustrated in FIG. 9. After the images have been taken while needle stage 200 is out of the way, the needle stage in installed, with a sterile needle 900. Using the controls labeled X control, Y control and Z control, the health professional moves the tip of needle 900 in alignment with a reference hole 902 in compression plate 104, and then presses a key or keys to indicate proper alignment. The procedure matches that in the current MultiCare system, but the target and reference coordinates have been obtained in the new way.

A second illustrative embodiment uses a scout view and two stereo views. Conceptually, it uses the scout image to identify the same first line in space, and uses the first stereo image to help identify a first vertical plane as discussed for the first embodiment. In addition, it uses a second stereo image to help identify a second vertical plane that passes through the abnormality and the x-ray origin at the time the second stereo image was taken, in a manner similar to that for identifying the first plane. The intersection of the two planes defines a second line in space. The intersection of the two lines, or their closest approach if they do not intersect, helps define the location of the abnormality or target in space. Alternatively, the second line is parallel to the plane of the compressed breast and its location in space can helps define needle penetration depth. Still alternatively, each pair of the scout image and a different stereo image can identify a target point, and a final target point can be selected based on the initially calculated target points, e.g., at a geometric center of the initial targets.

In the second example, the information from the second stereo image also is solely one-dimensional information. The y-value in the coordinate system of the second stereo view also can come from the y-value selected on the scout view, so that only an x-value need be taken from the second stereo view. The x-value from the second stereo view, the y-value from the scout view and the known distance from the x-ray origin to the image plane define a second vertical plane that intersects the origin position for the second stereo view and the projection of the abnormality or target onto the image plane of the second stereo view. These two points can be used in the same way as points 320 and 326 in the equation for the plane and the computer program described above to compute a location of the abnormality or target. Alternatively, the information regarding the two planes can be processed through known trigonometric relationships to yield information defining the line of intersection of the two planes.

If the locations of the target or abnormality calculated from each of the two planes coincide, or if the two lines intersect, then the location of interest is unambiguously established. If they do not, then the least distance between them can be found to help establish a single target location. If two three-dimensional locations for the target are found, the midpoint between them can be selected as the single target. If two lines are found, then a process can be carried out to find the least distance between them, for example using a known solution for this mathematical problem. One such solution is illustrated in Appendix A to this patent specification. A similar process is carried out in the current MultiCare system; however for two lines identified in a different way.

A third preferred embodiment adds an improvement to both the second example (that uses information from a scout view and each of two stereo views in the new way) and to the known approach of marking the location of an abnormality on each of two stereo images (i.e., deriving two-dimensional information from each). Conceptually, the known system calculates the position in space of a first line that passes through the abnormality and the x-ray origin when the first stereo image is taken, and the position in space of a second line passing through the abnormality and the x-ray origin when the second stereo image is being taken. If the two lines do not intersect, their closest approach defines the location of the abnormality for guiding the needle. However, in the known system the health professional may not be aware that the lines do not intersect, or of the distance between the non-intersecting lines. In order to overcome this and enable the health professional to make use of such information, the third preferred embodiment adds a live display or readout of relevant information. Preferably this live readout is displayed while the health professional is selecting a coordinate on an image. In the second example of the new approach, a display of information related to the spacing between two initially selected target locations, or between the two lines (one from the scout view and another from the intersection of the two vertical planes), can similarly guide the health professional.

The least distance between the points or lines can be important information for the health professional selecting coordinates in the displayed images. In accordance with the disclosure, this or related information is made available in a live display that enables the health professional to make a new selection of a coordinate accordingly, or to take some other action. Window 508 at monitor 206 in FIG. 5 shows such information dynamically during the coordinate selection process. For example, window 508 displays the least distance in appropriate units, such as millimeters, and may display additional information, such as the orientation of a vector along that distance. Based on this display, the health professional can make a new selection of a coordinate, to thereby affect the least distance, and can observe the result of the modification essentially instantaneously, with little or no perceived delay. For example, after initial selections of xy coordinates on the scout image and an x-coordinate on a stereo images the health professional may decide to alter the selection in a way that would reduce the least distance. To this end, a new selection may be made of an x-coordinate on a stereo image, the effect on the displayed least distance observed, another selection made, etc., until the health professional is satisfied with the result. Similarly, the health professional may modify the selection of a coordinate on the other stereo image, and/or the coordinates on the scout image to the same end, and may alternate between making new selections on the several images until satisfied, or can take some other action.

Similar new selections of coordinates from two stereo images may be carried out by providing a display 508 of the least distance in a system that is otherwise the same as the current MultiCare system, understanding that the least distance there is the distance between conceptual lines in space identified in a different way.

Any of the above processes for finding the position in space of a target and guiding the needle can be used for multiple targeting or multiple passes. Multiple targeting involves selecting targets that differ in depth into the breast, while multiple passes involves targets that differ in xy-coordinates (in the needle stage coordinate system, or in the plane of the scout image). The designation of multiple targets and multiple passes can be made in a manner analogous to that used in the current MultiCare system but based on an initial selection of a target 314 made in the new way.

While the x-value in a stereo image can be selected using the same input device (e.g., trackball 210 in FIG. 2) that selects xy values in the scout image, a currently preferred design is to use a separate input device that operates differently. One example is illustrated in FIG. 10 and comprises a slider 1000 that has a knob 1002 sliding left and right in a slot 1004. Knob 1002 is coupled through suitable electronics with the computer in workstation 204 to move marker line 500b (FIG. 5) to the left or right to match motion of knob 1002, and to indicate the selection of an x-value in a stereo image when knob 1002 is pressed down or input device 1000 is manipulated manually in some other way. Another input device may be used instead, such as the left-right arrow keys of keyboard 208, or another electro-mechanical device or set of devices. Slider 1000, or an alternative device, can be mounted at workstation 204 (FIG. 2) near trackball 210, for example to the right of the trackball as seen in FIG. 2.

The embodiments disclosed in this patent specification may also be practiced while the patient's upper body is in the upright position, with the patient, for example, seated or standing. As seen in FIG. 11, the apparatus used for this embodiment has an x-ray source that points down and is mounted on a C-arm that pivots about a vertical axis with the breast platform and the movable compression plate that are both aligned horizontally to allow for x-raying of the breast from above. Such apparatus, suitable for modifications to enable it to practice embodiments described herein, is commercially available from the assignee of this patent application, under the trade name Lorad StereoLoc. FIG. 11 designates elements that correspond to components of FIGS. 1 and 2 using the same reference numbers but with a prime (′).

While specific examples of the new approach and new system have been described in detail above, it should be clear that modifications, variations and additions would be apparent to persons skilled in the relevant technology on the basis of the disclosure in this patent specification. Accordingly, the scope of this patent specification is not limited to those specific examples by encompasses such modifications and additions to the extent allowed by the appended claims.

1. A method comprising:
taking a first x-ray view and a second x-ray view of a patient's breast compressed and immobilized between a breast platform and a breast plate having a biopsy opening, using a digital x-ray receptor, each of said views being taken from a different angle relative to the breast platform;
displaying a first image derived from the first view, selecting a location on the first image with a target symbol, and computer-generating a pair of coordinates related to the selected location;
computer-processing the pair of coordinates to generate information that defines a first conceptual line in space passing through a target in the breast related to the selected location in the first image;
displaying a second image derived from said second view, selecting a first marker line symbol on the second image, and computer-generating a first single coordinate related to the selected first marker line symbol on the second image;
computer-processing the first single coordinate to define a first conceptual plane that passes through the breast and intersects said conceptual line at a first intersection; and
controlling a needle guidance stage relative to the breast in accordance with positional information related to said first conceptual line and first conceptual plane.

2. A method comprising:
taking a scout x-ray view and a first stereo x-ray view of a compressed and immobilized breast of a patient using an x-ray receptor;
displaying a scout image derived from the scout view, selecting a location on the scout image with a target symbol, and computer-generating a pair of coordinates related to the selected location;
computer-processing the pair of coordinates to derive information related to a first conceptual line in space that passes through a target in the breast related to the selected location in the scout image;
displaying a first stereo image derived from said first stereo view, selecting a first marker line symbol on the stereo image, and computer-generating a first single coordinate related to the selected first marker line symbol on the first stereo image;
computer-processing the first single coordinate to derive information related to a first conceptual plane that passes through said target in the breast and intersects said conceptual line at a first intersection; and
controlling a needle guidance stage relative to the breast in accordance with positional information related to said first conceptual line and first conceptual plane.

3. A method as in claim 2 including taking a second stereo x-ray view, displaying a second stereo image derived from the second stereo view, selecting a second marker line symbol on the second stereo image, computer-generating a second single coordinate related to the selected second line symbol, computer-processing the second single coordinate to derive information related to a second conceptual plane that passes through said target in the breast and intersects said first conceptual line at a second intersection, wherein said controlling step comprises controlling the needle guidance stage relative to the breast in accordance with positional information regarding each of said conceptual line, first conceptual plane and second conceptual plane.

4. A method as in claim 3 in which said first and second conceptual planes intersect at a second conceptual line, and said controlling of the needle guidance stage in accordance with positional information regarding the first conceptual line and the first and second conceptual planes comprises controlling the needle guidance stage in accordance with positional information regarding the first and second conceptual lines.

5. A method as in claim 4 including computer processing positional information regarding said first and second conceptual lines to find a least distance between the first and second conceptual lines, and providing a live display of information regarding said least distance,

6. A method as in claim 5 in which the controlling comprises controlling the needle guidance stage in accordance with positional information regarding said least distance.

7. A method as in claim 6 including selecting at least one of a new location on the scout image and a new marker line symbol on at least one of the stereo images and computer-processing information related to the new selection, wherein making the new selection comprises using information from said live display of said least distance.

8. A method as in claim 7 in which making new selections comprises making new selections to reduce said least distance.

9. A method as in claim 8 comprising inputting information regarding additional targets and computer-processing said information to provide information for controlling the needle guidance stage in relation to said targets.

10. A method as in claim 2 comprising inputting information regarding additional targets and computer-processing said information to provide information for controlling the needle guidance stage in relation to said targets.

11. A method as in claim 10 wherein at least the upper body of the patient is prone while said x-ray views are taken.

12. A method as in claim 10 wherein the upper body of the patient is upright while said x-ray views are taken.

13. A method comprising:
taking a scout x-ray view and one or more stereo x-ray views of a compressed and immobilized breast of a patient using a digital x-ray receptor to receive x-rays passing through the breast;
displaying a scout image derived from the scout view and one or more stereo images derived from respective one or more stereo views, on a computer monitor;
selecting a location on the displayed scout image with a manually controlled first symbol displayed on the scout image;
computer-generating two-dimensional information regarding the selected location on the scout image;
selecting a plane crossing at least one of the one or more stereo images with a second symbol displayed on the at least one stereo image;
computer-generating one-dimensional information regarding the selected plane;
computer-processing the two-dimensional information from the scout image and the one-dimensional information from the at least one stereo image to produce three-dimensional information regarding a target in the breast related to both said two-dimensional and said one-dimensional information; and
providing information regarding the target to a needle guidance stage and controlling the needle guidance stage accordingly.

14. A method as in claim 13 wherein at least the upper body of the patient is prone while said x-ray views are taken.

15. A method as in claim 13 wherein the upper body of the patient is upright while said x-ray views are taken.

16. A method as in claim 13 in which the step of selecting a plane comprises selecting a plane from only a single stereo image.

17. A method as in claim 13 in which the step of selecting a plane comprises selecting a respective plane from each of at least two stereo images.

18. A method as in claim 17 wherein said computer-processing comprises producing three-dimensional information regarding at least two targets in the breast, and further comprises displaying information relating to the two targets and responding to a selection of at least one of a different location on the displayed scout image and a different plane on at least one displayed stereo image to change the displayed information relating to the two targets.

19. A method as in claim 15 comprising inputting information regarding additional targets and computer-processing said information to provide information for controlling the needle guidance stage in relation to said additional targets.

20. A method as in claim 19 wherein at least the upper body of the patient is prone while said x-ray views are taken.

21. A method as in claim 19 wherein at least the upper body of the patient is upright or while said x-ray views are taken.

22. A method of orienting a needle guidance stage relative to a compressed and immobilized breast of a patient and controlling needle depth for insertion into the breast using two-dimensional information regarding an area of interest seen in a scout view x-ray image of the breast and one-dimensional information regarding an area of interest seen in each of one or more stereo x-ray images of the breast, comprising:
using an x-ray source and a digital x-ray receptor to image a compressed and immobilized breast of a patient in a scout view and one or more stereo views;
displaying a scout image derived from the scout view and one or more respective stereo images derived from said one of said one or more stereo views, and deriving two-dimensional information regarding an area of interest seen on the scout view and one-dimensional information regarding an area of interest seen on said one or more stereo images;
computer-processing the two-dimensional information from the scout image and the one-dimensional information from each of the one or more stereo images to position a needle guidance stage relative to the breast and control needle depth for insertion into the breast.

23. A method as in claim 22, wherein the information derived from the scout view and each stereo view defines at least two targets or loci of targets in three-dimensional space in the breast, and said computer processing includes calculating and displaying a least distance between at least two of said targets or loci, and deriving new information regarding the areas of interest from at least one of said scout and stereo images and computer-processing the newly derived information to calculate a new set of targets or loci and at least one new, reduced least distance.

24. A method as in claim 22, including deriving two-dimensional information from at least one of the stereo views, and wherein said computer-processing of information from the scout image and the one or more stereo images comprises calculating at least two targets or loci of targets, and further calculating information regarding at least one least distance between said targets or loci and providing a display of results related to the least distance calculation.

25. A method as in claim 24 in which said display of results comprises information defining a current value of said least distance.

26. A method as in claim 25 in which said current value in the display of results changes in response to newly derived two-dimensional or one-dimensional information.

27. A method as in claim 26 wherein at least the upper body of the patient is prone while said x-ray views are taken.

28. A method as in claim 26 wherein at least the upper body of the patient is upright while said x-ray views are taken.

29. A method comprising:
taking two stereo x-rays views of a compressed and immobilized breast of a patient;
displaying two stereo images derived from said two stereo views;
marking a location on each displayed image related to an abnormality seen on each;
computer-generating two pairs of coordinates, each pair related to a respective one of the marks;
computer-processing the two pairs of coordinates to calculate positional information related to a pair of conceptual lines passing through the breast and to calculate a least distance between the lines;
providing a live display of information related to said least distance;
re-marking at least one of the displayed images to produce a new set of pairs of coordinates and using said new set in said computer-processing to calculate and display a new least distance, and selectively repeating said re-marking and computer-processing of new sets of pairs to reduce said least distance; and
using information regarding the conceptual lines and the currently calculated least distance to guide a needle stage relative to the breast.

30. A method as in claim 29 wherein at least the upper body of the patient is prone while said x-ray views are taken.

31. A method as in claim 29 wherein at least the upper body of the patient is upright while staid x-ray views are taken.

32. A system comprising:
a breast platform and a compression plate movable relative to each other to compress and immobilize a patient's breast between them;
a digital x-ray receptor at one side of the platform and plate, and an x-ray source at the other side, said receptor and source mounted for rotation about the platform to image the breast from multiple angles;
a needle guidance stage selectively positioned between the x-ray source and the breast and carrying a needle oriented transversely to a plane conforming to a main plane of the compressed breast;
a computer coupled with the x-ray receptor to receive x-ray exposure information therefrom for at least two different views of the breast taken at different angles and to generate image information for corresponding images;
a computer monitor coupled with said computer to receive said image information and display images derived therefrom;
at least one marking device coupled with said computer to selectively place a first marker at a location in a first one of said displayed images and a second marker at a line on a second one of said displayed images to mark a plane intersecting the image;
said computer responding to said marking to produce a coordinate pair related to the marked location on the first image but only a single coordinate related to the plane marked on the second image;
said computer processing said pair of coordinates from the first image and said single coordinate from the second image to calculate a location of a target in the breast and provide information for positioning the needle guidance stage relative to the breast and for depth of insertion of a needle into the breast.

33. A system comprising:
a patient table having an opening for a breast of a prone patient;
a breast platform and a compression plate movable relative to each other under the table to compress and immobilize a breast protruding downwardly through said opening;
a digital x-ray receptor at one side of the platform and plate and an x-ray source at the other side, said receptor and source mounted for rotation about the platform and plate to image the breast in a scout view and one or more stereo views;
a needle guidance stage selectively positioned between the x-ray source and the breast and carrying a needle oriented transversely to a plane conforming to a main plane of the compressed breast;
a computer coupled with the x-ray receptor to receive x-ray exposure information therefrom for a scout view and one or more stereo view of the breast and to generate image information for corresponding scout and one or more stereo images;
a computer monitor coupled with said computer to receive said image information and display a scout image and one or more stereo images derived therefrom;
at least one marking device coupled with said computer to place a first marker at a location in the displayed scout image, and to place a second marker in each of the displayed one or more stereo images to mark on each a respective plane intersecting the respective stereo image;
said computer responding to said marking to produce a coordinate pair related to the marked location on the scout image but only a single coordinate related to each marked intersecting plane;
said computer processing said pair of coordinates from the scout image and said single coordinate related to one or more of said planes to calculate a three dimensional position of at least one target in the breast and provide information for positioning the needle guidance stage relative to the breast and for depth of insertion of a needle into the breast.

34. A system as in claim 33 in which said computer is configured to respond to said marking to calculate at least two positions or loci of positions of a target in the breast, and further to calculate at least one least distance between said positions or loci in the breast and provide information related thereto to said monitor, and wherein said monitor displays information regarding said at least one least distance in a live display.

35. A system as in claim 34 in which said computer is configured to respond to new marks on at least one of said scout and stereo images to recalculate said at least one least distance and display recalculated results related thereto on said monitor.

36. A system as in claim 34 in which said computer is configured to provide said information for positioning the needle guidance system and for selecting needle depth based in part on said calculated least distance.

37. A system comprising:
a breast platform and a compression plate movable relative to each other under the table to compress and immobilize a breast of a patient;
an x-ray receptor at one side of the platform and plate and an x-ray source at the other side, said receptor and source mounted for rotation about the platform and breast to image the breast in views taken at different angles;
a needle guidance stage selectively positioned between the x-ray source and the breast and carrying a needle pointing to the compressed breast;
a display for x-ray images derived from said views;
a marking device for selecting and marking a location of interest on each of the images;
a computer coupled with the marking device and responsive to said selection of locations to produce two pairs of coordinates, each pair related to a respective selected locations;
said computer processing said two pairs of coordinates calculate, in three dimensions, at least two locations in the breast and to calculate at least one least distance between said locations in the breast;
a display device providing a live display of information related to said at least one least distance; and
said computer being configured to provide information to said needle guidance stage, based in part on information relating to said at least one least distance, for positioning the needle guidance stage relative to the breast and for a depth of insertion of a needle into the breast.

38. A system as in claim 37 in which said computer is configured to respond to a new selection of a location on at least one of said images to recalculate said at least one least distance and display recalculated results on said display of results.

39. A system as in claim 37 including a table supporting at least the upper body of the patient in a prone position.

40. A system as in claim 37 in which said platform supports the breast while at least the upper body of the patient is upright.
